(19) United States
(12) Patent Application Publication
(10) Pub. No.: US 2021/0022693 A1
(43) Pub. Date: Jan. 28, 2021

(54) METHODS FOR DETECTING POSITIONAL MOVEMENT OF ORTHOPEDIC IMPLANTS

(71) Applicant: Rowan University, Glassboro, NJ (US)

(72) Inventors: Erik Brewer, Conshohocken, PA (US); Hanna Dietrich, Ocean City, NJ (US); Joseph DiFranceisco, Sewell, NJ (US); Ann DiGuglielmo, Somerdale, NJ (US); Miguel Isaga, Delran, NJ (US); Luke Austin, Haddonfield, NJ (US); Hamid Seyyedhosseinzadeh, Mullica Hill, NJ (US); Mary Staehle, Moorestown, NJ (US)

(21) Appl. No.: 16/966,613

(22) PCT Filed: Feb. 13, 2019
(86) PCT No.: PCT/US2019/017861

§ 371 Date: Jul. 31, 2020

Related U.S. Application Data
(60) Provisional application No. 62/630,615, filed on Feb. 14, 2018.

(51) Int. Cl. A61B 6/12; A61B 34/20; A61F 2/32; A61F 2/36; A61F 2/38; A61F 2/40; A61F 2/42

(52) U.S. Cl. CPC A61B 6/12; A61B 34/20; A61F 2/32; A61F 2/36; A61F 2002/3008; A61F 2/40; A61F 2/4202; A61B 2034/2072; A61F 2/38

(57) ABSTRACT

Methods for detecting positional movement of orthopedic implants are described herein. In one embodiment, the method may include receiving a two-dimensional image defining an image plane, the two-dimensional image capturing a reference marker received within the bone and an orthopedic implant received within a bone, calculating a first angle of the reference marker relative to the image plane based on previously stored dimensions of the reference marker, calculating a second angle of the orthopedic implant relative to the image plane based on previously stored dimensions of the orthopedic implant, comparing the first angle to the second angle to calculate a current angle of rotation, and comparing the current angle of rotation to a previously calculated angle of rotation to detect rotation of the orthopedic implant relative to the bone.

### CROSS-REFERENCE TO RELATED APPLICATION(S)

This application claims priority to U.S. Provisional Patent Application Ser. No. 62/630,615, filed Feb. 14, 2018. The entire content of this application is hereby incorporated by reference herein.

### BACKGROUND OF THE INVENTION

There are approximately 2.5 million individuals living with hip replacement implants in the United States alone, with an additional 300,000 new hip replacement surgeries conducted each year on average. However, in many arthroplasty patients, the joint implant may loosen over time (e.g., rotationally, translationally, or a combination thereof). Should the joint implant loosen to a severe degree (e.g., total implant failure), the patient may need to undergo major revision surgeries to correct the loosening.

### SUMMARY OF THE INVENTION

One aspect of the invention provides a computer-implemented method for detecting rotation of orthopedic implant. The method includes: receiving a two-dimensional image defining an image plane, the two-dimensional image capturing a reference marker received within a bone and an orthopedic implant received within the bone; calculating a first angle of the reference marker relative to the image plane based on previously stored dimensions of the reference marker; calculating a second angle of the orthopedic implant relative to the image plane based on previously stored dimensions of the orthopedic implant; comparing the first angle to the second angle to calculate a current angle of rotation; and comparing the current angle of rotation to a previously calculated angle of rotation to detect rotation of the orthopedic implant relative to the bone.

This aspect of the invention can include a variety of embodiments. In one embodiment, the two-dimensional image is a radiograph.

In one embodiment, the reference marker is isolated from the orthopedic implant. Additionally or alternatively, the reference marker is a screw. Additionally or alternatively, the screw includes a cylindrical portion. In one embodiment, the cylindrical portion is an outer profile of a head of a screw. In one embodiment, the screw was installed in a bore utilized for mounting a computer-assisted navigation system during installation of the orthopedic implant.

In one embodiment, the orthopedic implant is selected from a consisting of a hip implant, a femoral implant, a knee implant, an ankle implant, and a should replacement. Additionally or alternatively, the orthopedic implant can include a radiopaque marker having a cylindrical profile.

### BRIEF DESCRIPTION OF THE DRAWINGS

For a fuller understanding of the nature and desired objects of the present invention, reference is made to the following detailed description taken in conjunction with the accompanying drawing figures wherein like reference characters denote corresponding parts throughout the several views.

FIGS. 1A and 1B depict a joint implant and reference implant for a hip replacement procedure according to embodiments of the invention.

FIG. 2 depicts scenarios of joint implant loosening according to an embodiment of the invention.

FIGS. 3-5 depict a screw assembly according to an embodiment of the invention

FIG. 6 depicts a 3-dimensional visualization of a joint implant according to an embodiment of the invention.

FIG. 7 depicts a process for detecting rotation of an orthopedic implant according to an embodiment of the invention.

FIG. 8 depicts a screw (highlighted with an ellipse) utilized for a computer-assisted navigation (CAN) system for joint replacement surgery, the bore for which can be utilized for a reference implant according to an embodiment of the invention.

### DEFINITIONS

The instant invention is most clearly understood with reference to the following definitions.

As used herein, the singular form “a,” “an,” and “the” include plural references unless the context clearly dictates otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used in the specification and claims, the terms “comprises,” “comprising,” “containing,” “having,” and the like can have the meaning ascribed to them in U.S. patent law and can mean “includes,” “including,” and the like.

Unless specifically stated or obvious from context, the term “or,” as used herein, is understood to be inclusive.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

### DETAILED DESCRIPTION OF THE INVENTION

In certain aspects, the invention provides a computer-implemented method for detecting rotation of an orthopedic implant. In other aspects, the invention provides for a device for detection of rotation of an orthopedic implant.

### Orthopedic Implant Detection Device

Joint replacement procedures are common surgical procedures conducts throughout the world. These types of procedures provide mobility and pain mitigation to a multitude of individuals. However, there is substantially high risk that joint replacements, once implanted, will fail at some point in time. Conventional techniques for detecting joint replacement failure are limited to detecting failure after the failure has already occurred, or are cost prohibitive due to their technological complexity and/or governmental regulations.

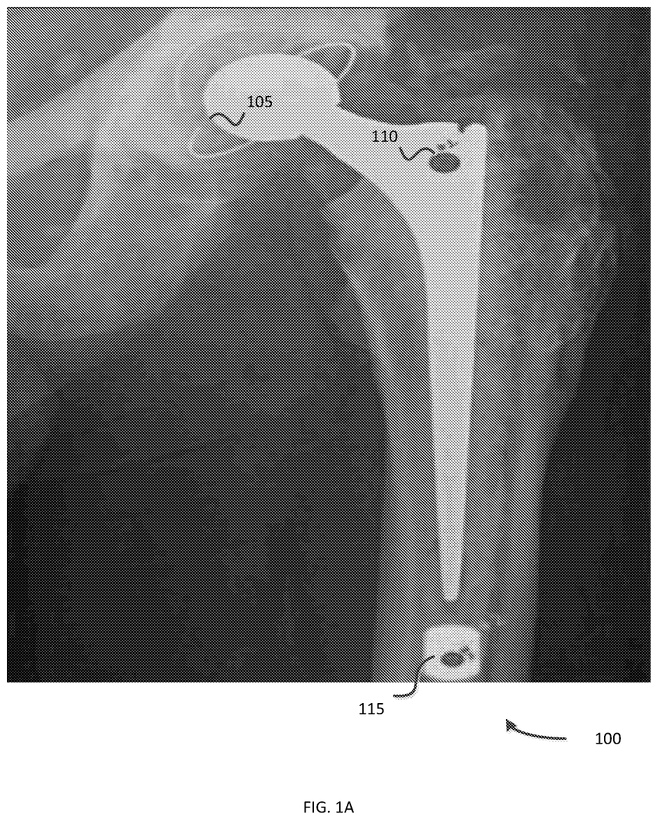

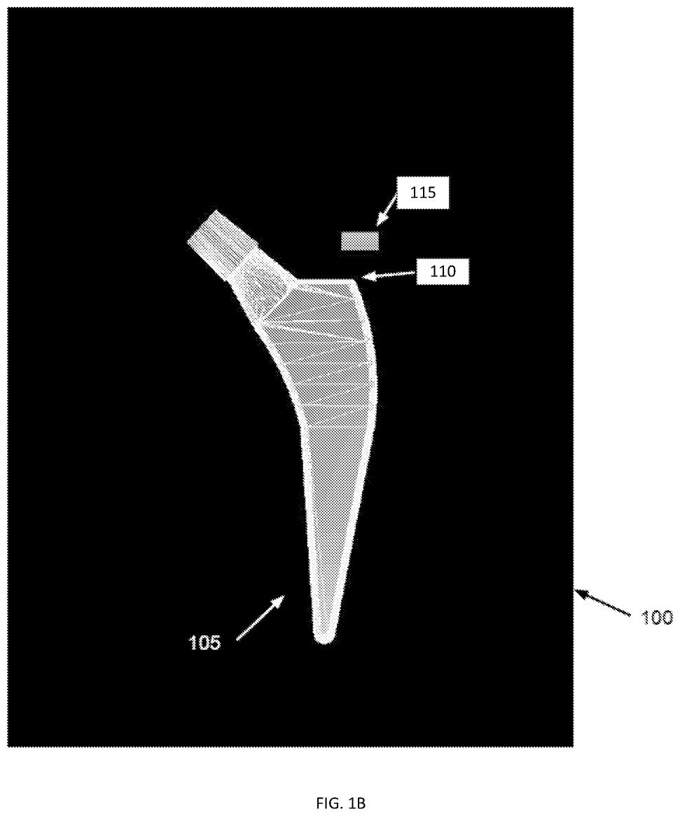

Referring to FIGS. 1A and 1B, one embodiment of the invention provides for a novel orthopedic implant detection device assembly 100 comprising a joint implant 105 and a reference implant 110. The joint implant 105 may also include a reference point 115 affixed to the joint implant 105. A change in the relation between the reference implant 110 and the reference point 115 can provide information corresponding to a change in the positional relationship between the joint implant and the joint socket which the joint implant 105 is implanted in. This, in turn, may provide information as to whether the joint implant 105 is loosening in the joint socket. If caught prior to total implant failure, the patient may undergo a less invasive, less severe surgery in repairing the joint replacement, thereby mitigating the risks and issues associated with surgical repair of total implant failure.

### Joint Implant

The joint implant 105 may include a standard joint implant used for joint reconstructive/replacement surgeries. The joint implant 105 can be formed to fit the socket of the designated joint, and may be formed using conventional methods. Further, the joint implant 105 can be formed of various composition, including metal, plastic, or ceramic. While examples provided in the figures, such as in FIGS. 1A and 1B, illustrate a hip joint implant assembly 100, where the implant detection device may be constructed for any joint type, including shoulder, hip, knee, ankle, wrist, elbow, etc.

The joint implant 105 may include at least one reference point 110. The reference point may in some cases be attached to the joint implant 105; however in other cases the reference point 110 may be a distinguishable portion of the joint implant 105. For example, the reference point 110 may be a distinguishable location on the edge of the joint implant 105.

The reference point 110 can be visualized using differences in radiopacity (e.g., when visualized through x-ray). For example, the reference point 110 can be more radiopaque or less radiopaque than the adjacent implant 105. In some embodiments, the reference point 110 is a void in the implant 105.

The reference point can also be a combination of points along the geometry of the implant 105, the difference between which in a 2-D plan will change as the implant 105 is rotated.

In conjunction with the reference implant 115 and, in some embodiments, other reference points 110, the computer-implemented method provided below may be able to determine translational and/or rotation movement of the joint implant 105.

### Reference Implant

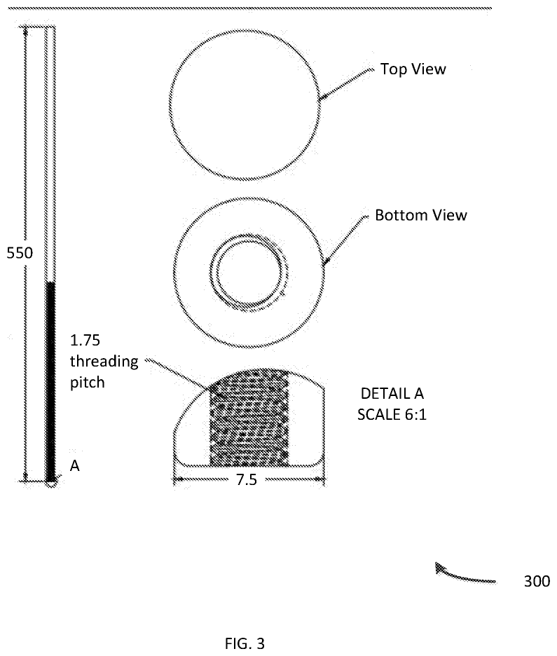

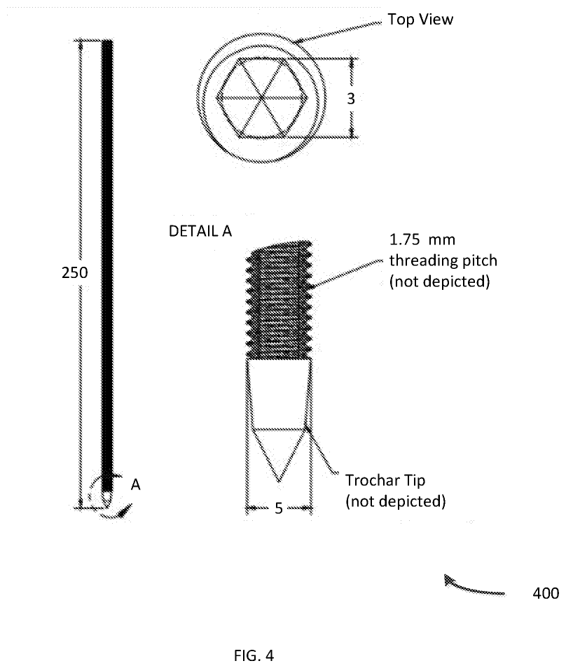

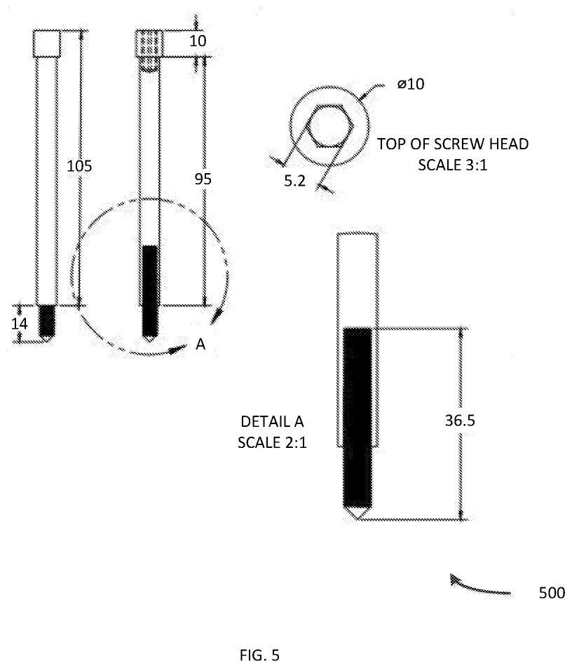

The orthopedic implant detection device assembly 100 may also include the reference implant 115. FIGS. 3-5 illustrate various perspectives of a reference implant.

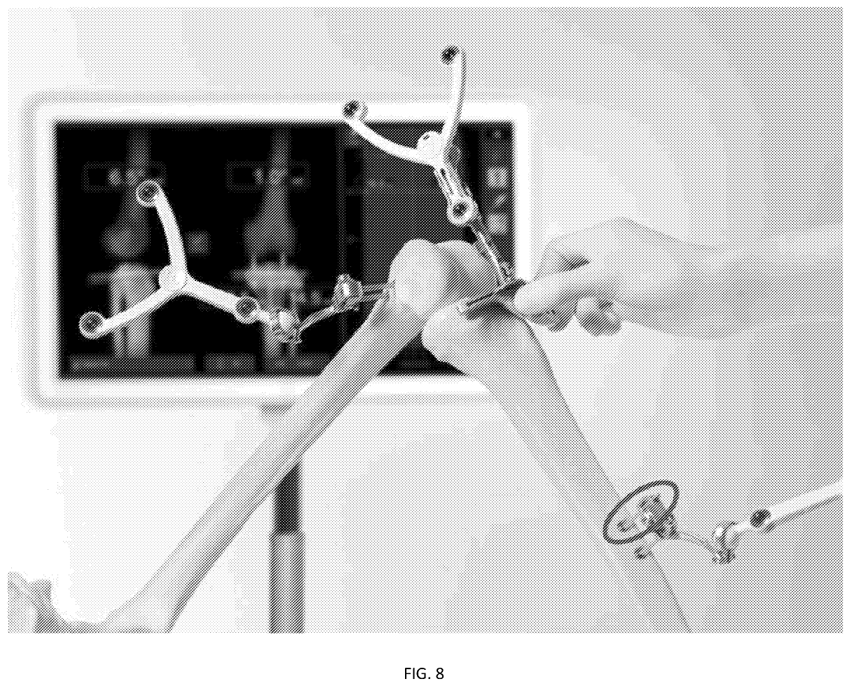

The reference implant 115 may be inserted during the initial arthroplasty surgery. In some embodiments, the reference implant 115 is placed in a hole created for placement of Schanz screws for a computer-assisted navigation system (CAN) used in arthroplasty surgeries as depicted in FIG. 8 in which one of the potential screw placements is highlighted by an oval. This may allow for no additional surgical procedures for the patient.

The reference implant 115 may be inserted into a connecting bone of the joint implant 105. For example, in the case of a hip replacement, the reference implant 115 may be inserted into the femur, where the joint implant 105 may be inserted into (e.g., inserted distally into the femur). Thus, the reference implant 115 may remain stationary in the bone. Additionally or alternatively, the location for inserting the reference implant 115 may be selected according to other factors, such as an area with a low immunological response and/or a low mechanical loading (e.g., the greater trochanter for hip implants).

### Exemplary Reference Implant Embodiment

An exemplary reference implant may include a telescoping screw system. The screw system may replace a screw (e.g., a Schanz screw) that may have been used for a computer-assisted navigation (CAN) system for joint replacement surgery. This screw replacement may require no additional surgery, as the CAN screw may already be implanted into the patient's bone for the CAN system. Subsequent to inserting the joint implant, the CAN screw may be removed from the bone, and the reference implant may be inserted in the vacated location where the CAN screw was located. The screw may have, for example, a 5 mm diameter, including threading with a 1.75 mm pitch. The screw may additionally or alternatively include a trocar tip. The screw length may be dependent upon the femur diameter (e.g., 25 mm in length may be typical). The screw may also exclude a screw head, and in some cases may include a hexagonal socket drive.

The telescoping screw system may also include multiple barrel nuts attached for the screw. A first barrel nut may be attached to the drive end of the screw. The first barrel nut may have a length of 500 mm, a diameter of 7.5 mm, and internal threading that matches the screw. The first barrel nut may be used for attachment of CAN system probes.

After surgery is complete, the first barrel nut may be removed, which may leave the screw securely in place. A second barrel nut with a shorter length than the first barrel nut (e.g., 10 mm) with a hexagonal socket drive may be attached to the screw. The second barrel nut and screw combination may then be drilled into the bone until the second barrel nut is flush with the bone. The separate composition of the second barrel nut may provide for a distinguishing feature in relation to the surrounding bone composition when viewed via radiography.

### Implant Movement Detection

The existence of rotational or translational movement of the joint implant may be determined based on the positioning of the joint implant relative to the reference implant. This determination may be made for any readily available implant device, even for joint implants that have been implanted previously without a reference implant (e.g., the reference implant may be inserted at a later time than the joint implant). This determination may be made with radiograph imaging (e.g., a two-dimensional x-ray) along with data analysis software (e.g., IMAGEJ™ software). Several different movement detection methods may be utilized by data analysis software in order to make this determination. For example, in a centroid method, an original radiograph image be taken shortly after inserting the joint implant and the reference implant. The original radiograph image may be uploaded (e.g., via a computer) and stored in a database (e.g., as a .jpeg file). The analysis software may identify at least one coordinate of a predefined centroid of the joint implant. The centroid may be defined through the data analysis software (e.g., the center of the widest visible portion of the implant), or the centroid may be defined manually.

Additional radiographs may be taken of the implant over time to determine whether the implant joint has loosened from the joint socket. A second radiograph may be taken of the implant and uploaded to the database as described above. The data analysis software may identify an outline of the joint implant and, based on the identified outline, determine a change in location for the centroid. A change in location for the centroid using this method can identify a translational change, a rotational change, or a combination thereof, where the reference implant is utilized as a reference from the original radiograph as to how the joint implant was originally situated in the joint socket.

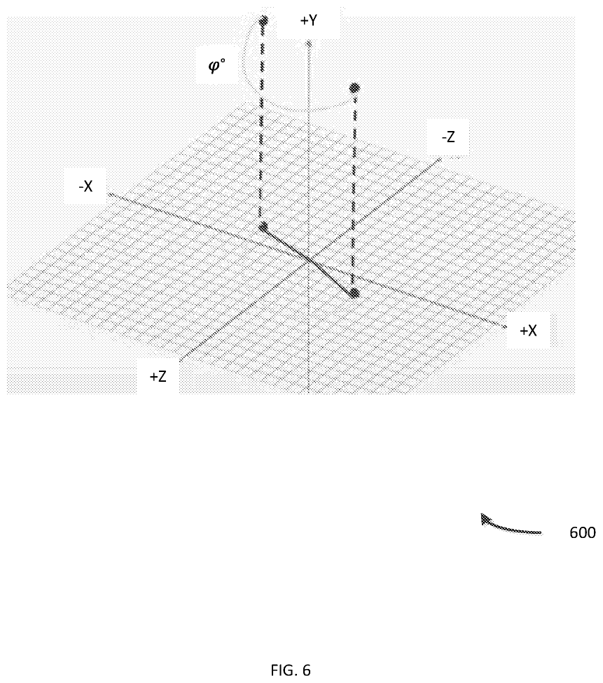

Another method for detecting joint implant movement is a template method. In the template method, an original radiograph is taken of the implant and uploaded as described above. The original radiograph in this method acts as a template, where coordinates are identified of the joint implant, without prior knowledge of the particular implant. Additional radiographs are taken of the implant site over time, and these additional radiographs are overlaid atop the two-dimensional outline of the joint implant and reference implant. The scale of the radiographs are then calibrated by matching the size of the reference implants in both the original and additional radiographs. The additional radiograph is then translated and rotated, via the data analysis software, until the implant assembly of the additional radiograph matches the implant assembly shown in the original radiograph. Once matched, the data analysis software may then calculate, by analyzing the change in coordinate positions, the deviation the implant assembly of the additional radiograph experienced relative to the implant assembly of the original radiograph. The template method may allow for a patient-specific and implant-neutral approach to detecting implant movement, since the movement of the joint is tracked from an original radiograph taken. With either detection method discussed above, the data analysis software generates a 3-dimensional image based on the change between the coordinate positions of the radiographs. FIG. 6 illustrates a 3-dimensional visualization 600 of rotational movement for a joint implant. As can be seen, there is an x-direction, a y-direction, and a z-direction. If, for example, the y-direction elongation for coordinates between radiographs remains relatively constant, then the joint implant centering and the angle between the join implant and the attached bone are relatively constant as well. If a change is determined in the x-direction, then the reference implant may be rotated relative to the attached bone.

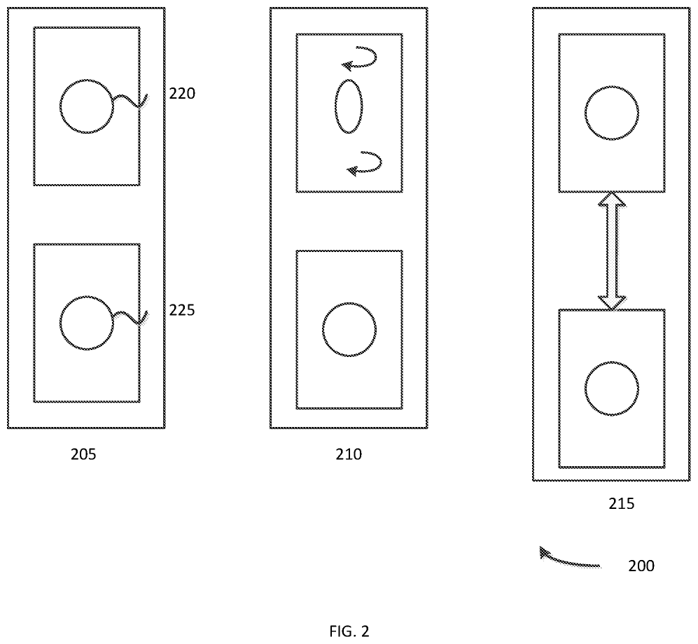

Another illustration of coordinate movements can be seen in FIG. 2. The first scenario 205 may be described as the implant assembly as being in a normal position, where the reference point 220 and the reference implant 225 are in the originally inserted positions. The second scenario 210 may be described as the implant assembly as having experienced rotation movement, where the reference point 220 of the joint implant has rotated to an so as to appear elliptical when viewed in a 2-D plane, even though the feature remains circular or cylindrical. The third scenario 215 may be described as having experienced translational movement, as the distance between the reference point 220 of the joint implant and the coordinate of the reference implant 225 has increased relative to the normal position.

The data analysis software may then utilize the coordinates and the dimensions of the joint implant to calculate any rotational movement. For example, the data analysis software may calculate the rotation of a specific coordinate about the y-axis based on a transformation matrix. An example of a transformation matrix is provided below:

$$[ \begin{matrix}{X}_{1} \\ {Y}_{1} \\ {Z}_{1}\end{matrix} ] = [ \begin{matrix}cos ϕ & 0 & sin ϕ \\ 0 & 1 & 0 \\ - s in ϕ & 0 & cos ϕ\end{matrix} ] \times [ \begin{matrix}{X}_{0} \\ {Y}_{0} \\ {Z}_{0}\end{matrix} ]$$

where X₁, Y₁, and Z₁ are transformed coordinates; φ is the angle of rotation around the y-axis, and X₀, Y₀, and Z₀ are the initial coordinates. Thus, the matrix equations may be represented as the following set of equations:

X₁=X₀ cos θ+Y₀(0)+Z sin θ

Y₁=X₀(0)+Y₀(1)+Z(0)

Z₁=X₀(−sin θ)+Y₀(0)+Z₀ cos θ

The data analysis software may solve these equations to determine the rotational and translational movement of the joint implant.

Both the centroid method and the template method allow for detection of the loosening of a join implant prior to total implant failure. Furthermore, as the detection method relies on simple, 2-D radiographs for early detection, the costs associated with this detection method are significantly reduced (e.g., as compared to 3-D imaging techniques).

### Exemplary Workflow

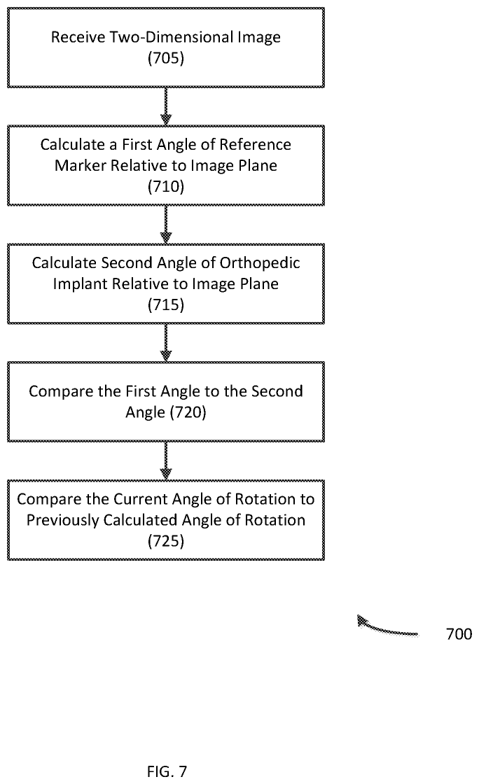

FIG. 7 describes an exemplary workflow 700 for detecting positional movement of a joint implant, in accordance with embodiments of the current invention. The workflow 700 may include a joint implant assembly, such as joint assembly 100 as described above with reference to FIGS. 1A and 1B.

At Step 705, a computer may receive a two-dimensional image defining an image plane. The two-dimensional image may capture a reference marker received within a bone and an orthopedic implant received with the bone. At Step 710, the computer may calculate a first angle of the reference marker relative to the image plane based on previously stored dimensions of the reference marker. At Step 715, the computer may calculate a second angle of the orthopedic implant relative to the image plane based on previously stored dimensions of the orthopedic implant. At Step 720, the computer may compare the first angle to the second angle to calculate a current angle of rotation. At Step 725, the computer may compare the current angle of rotation to a previously calculated angle of rotation to detect rotation of the orthopedic implant relative to the bone.

### EQUIVALENTS

Although preferred embodiments of the invention have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference.

1. A computer-implemented method of detecting rotation of an orthopedic implant, the computer-implemented method comprising:
receiving a two-dimensional image defining an image plane, the two-dimensional image capturing:
a reference marker received within a bone; and
an orthopedic implant received within the bone;
calculating a first angle of the reference marker relative to the image plane based on previously stored dimensions of the reference marker;
calculating a second angle of the orthopedic implant relative to the image plane based on previously stored dimensions of the orthopedic implant;
comparing the first angle to the second angle to calculate a current angle of rotation; and
comparing the current angle of rotation to a previously calculated angle of rotation to detect rotation of the orthopedic implant relative to the bone.

2. The computer-implemented method of claim 1, wherein the two-dimensional image is a radiograph.

3. The computer-implemented method of claim 1, wherein the reference marker is isolated from the orthopedic implant.

4. The computer-implemented method of claim 1, wherein the reference marker is a screw.

5. The computer-implemented method of claim 4, wherein the screw includes a cylindrical portion.

6. The computer-implemented method of claim 5, wherein the cylindrical portion is an outer profile of a head of the screw.

7. The computer-implemented method of claim 4, wherein the screw was installed in a bore utilized for mounting a computer-assisted navigation system during installation of the orthopedic implant.

8. The computer-implemented method of claim 1, wherein the orthopedic implant is selected from the group consisting of: a hip implant, a femoral implant, a knee implant, an ankle implant, and a shoulder replacement.

9. The computer-implanted method of claim 1, wherein the orthopedic implant comprises a radiopaque marker having a cylindrical profile.
